(19) United States
(12) Patent Application Publication
(10) Pub. No.: US 2022/0076785 A1
(43) Pub. Date: Mar. 10, 2022

(54) METHOD FOR ACQUIRING INTRACELLULAR DETERMINISTIC EVENT, ELECTRONIC DEVICE AND STORAGE MEDIUM

(71) Applicant: PHIL RIVERS TECHNOLOGY, LTD., Beijing (CN)

(72) Inventors: Gang NIU, Beijing (CN); Yanhui FAN, Beijing (CN); Qiangzu ZHANG, Beijing (CN); Chunming ZHANG, Beijing (CN); Guangming TAN, Beijing (CN); Zhendong FENG, Beijing (CN)

(21) Appl. No.: 17/417,018

(22) PCT Filed: Dec. 21, 2018
(86) PCT No.: PCT/CN2018/122787

§ 371 Date: Jun. 21, 2021

(51) Int. Cl. G16B 20/50; G16B 5/20

(52) U.S. Cl. CPC G16B 20/50; G16B 5/20

(57) ABSTRACT

The present application discloses a method for acquiring intracellular deterministic event, an electronic device and a storage medium, the method includes: acquiring several mutant genes of a detected object; acquiring driving force information of each of the several mutant genes for changes of each gene in a predetermined genome; acquiring driving force information of the several mutant genes for changes of each gene in the predetermined genome according to the acquiring driving force information of each of the several mutant genes for changes of each gene in the predetermined genome; and determining at least onepredetermined type of intracellular deterministic event information of the detected object according to the driving force information of the several mutant genes for changes of each gene in the predetermined genome.

### CROSS REFERENCE TO RELATED APPLICATIONS

The present application is a 35 U.S.C.§371 national phase application of PCT Application Ser. No. PCT/CN2018/122787 filed on Dec. 21, 2018, the entire contents of which are incorporated herein by reference.

### TECHNICAL FIELD

The present application relates to the biotechnology, and more particularly to a method for acquiring intracellular deterministic event, an electronic device and a storage medium.

### BACKGROUND

Breast cancer is one of the most important threats to the health of women worldwide. There are approximately 1.3 million newly increased breast cancer cases and approximately 500,000 deaths worldwide each year.Taking the statistics data of China in 2015 and the United States in 2018 as examples, the incidence of breast cancer in the two countries ranked first among all cancers in women, and the mortality rate ranked fifth and second respectively, and as of the statistical time, the total number of surviving patients exceeded 260,000. On average, every woman has a 12% chance of getting breast cancer in her lifetime. Early prevention, early detection, and early treatment have proven to significantly improve the prognosis of breast cancer patients in a number of retrospective studies, especially triple-negative breast cancer with early onset, poor prognosis, and unknown mechanism.Therefore, there is an urgent need for a comprehensive assessment of the risk of breast cancer using the data and information that can be collected during the asymptomatic period, and the germline genetic information is a good choice.

### TECHNICAL PROBLEM

An object of the present application is to provide a technical solution for acquiring definite intracellular events using germline genetic information that can be collected during asymptomatic period.

### SUMMARY

A first aspect of the present application provides a method for acquiring intracellular deterministic event, executed by an electronic device, including:

S11: acquiring several mutant genes of a detected object;

S12: acquiring driving force information of each of the several mutant genes for changes of each gene in a predetermined genome;

S13: acquiring driving force information of the several mutant genes for changes of each gene in the predetermined genome according to the acquiring driving force information of each of the several mutant genes for the changes of each gene in the predetermined genome; and

S14: determining at least onepredetermined type of intracellular deterministic event information of the detected object according to the driving force information of the several mutant genes for the changes of each gene in the predetermined genome.

A further aspect of the present application provides an electronic device,including: a memory, a processor, and a program stored in the memory, the program is configured to be executed by the processor, and when the processor executes the program to implement the above mentioned method for acquiring intracellular deterministic event.

A further aspect of the present application provides a storage medium, storing a computer program, when the computer program is executed by a processor, to implement the above mentioned method for acquiring intracellular deterministic event.

### BENEFICIAL EFFECTS

In some embodiments of the present application, the germline genetic information that can be collected during the asymptomatic period is used to obtain intracellular deterministic event through the driving force information of the mutant gene of the detected object for changing the gene in the genome.

In some embodiments of the present application, all germline genetic information are used to comprehensively evaluate the basis of the overall characteristics of germline inheritance, so that it can cover the risks evaluation of various sporadic and familial genetic diseases (such as breast cancer) caused by germline inheritance, and the sensitivity of detecting individuals at risk is improved.

In some embodiments of the present application, germline variation features with discrete, high-dimensional, multi-correlated, and non-standardized can be projected to gene prediction expression features and signal pathway activity features with continuous range, relatively low-dimensional, and gradually converging correlation, it constructs a quantitative model that converts discrete qualitative data into continuous space, on the one hand, it retains the global features of the data, on the other hand, it becomes the basis of data-driven classification that correlates germline genetic information with other deterministic events in breast cancer (including but not limited to lymph node metastasis, age of onset and other pathophysiological characteristics).

In some embodiments of the present application, since the input source is a global germline rare mutation, the risk rating and clinical feature correlation of sporadic genetic breast cancer such as triple-negative breast cancer can be graded according to pathway activity, which fills up the gap in the coverage of the knowledge-driven approach based on gene panel and significantly reduces the false negative rate.

In some embodiments of the present application, the risk of disease can be correlated with other clinical, pathological, physiological, or behavioral related deterministic event features, so that the model can provide a basis for prognostic evaluation, early clinical intervention and management of patients based on germline genetic information.

### BRIEF DESCRIPTION OF DRAWINGS

In order to explain the technical solution of embodiments of the present application more clearly, the drawings used in the description of the embodiments will be briefly described herein below. Obviously, the drawings in the following description are some embodiments of the present application, and for persons skilled in the art, other drawings may also be obtained on the basis of these drawings without any creative work.

FIG. 1 is a schematic flowchart of a method for acquiring an intracellular deterministic event in accordance with an embodiment of the present application;

FIG. 2 is a schematic flowchart of a method for acquiring an intracellular deterministic event in accordance with another embodiment of the present application;

FIG. 3 is a schematic flowchart of a method for predicting a risk of suffering from disease in accordance with an embodiment of the present application;

FIG. 4 is a schematic structural diagram of an electronic device in accordance with an embodiment of the present application.

### DETAILED DESCRIPTION OF THE EMBODIMENTS

In order to enable those skilled in the art to better understand the technical solutions of the present application, the technical solutions in the embodiments of the present application will be further described in detail herein below in conjunction with the drawings. Obviously, the embodiments described are partial embodiments of this application, but not all of the embodiments. On the basis of the embodiments in this application, all other embodiments obtained by those skilled in the art without paying any creative work should fall within the protection scope of the present application.

The term “comprise/include” in the specification and claims of the present application and the above-mentioned drawings and any variations thereof are intended to cover non-exclusive inclusions. For example, a process, method or system, product or device that includes a series of steps or units is not limited to the listed steps or units, but optionally includes unlisted steps or units, or optionally also includes other steps or units inherent in these processes, methods, products or equipment. In addition, the terms “first”, “second” and “third” are used to distinguish different objects, rather than to describe a specific order.

In the present application, global germline genetic information refers to all genetic information derived from parents, encoded in the genomes of all normal cells developed from embryos, carried by individuals throughout their lives, and inherited to offspring through reproduction. The form includes but is not limited to genomic DNA sequence, epigenetic modification information, etc..

In the present application, an intracellular deterministic event refer to event characteristics ultimately produced through the interaction of various molecules in the organism based on known or unknown mechanisms that can be detected qualitatively or quantitatively by various methods, including but not limited to activation or inhibition of signaling pathway, changes in types and content of metabolites, the interaction mode, state and its interactome between biomolecules (including large molecules such as proteins/nucleic acids, and small molecules such as lipids/small molecule drugs/metabolites/inorganic metal ions,) polymer/cell/tissue organ structure and its changes, etc. In the present application, the intracellular deterministic event include gene expression that is genetically determined in the germline, signaling pathway activity, disease risk or resistance to breast cancer, and probability of occurrence of pathophysiological conditions related to breast cancer.

FIG. 1 shows a schematic flow chart of a method for acquiring an intracellular deterministic event according to an embodiment of the present application. The method may be executed by an electronic device and includes:

S11: acquiring several mutant genes belonging to a pre-determined genome of a detected object.

S12: acquiring driving force information of each of the several mutant genes for changes of each gene in the pre-determined genome.

S13: acquiring driving force information of the several mutant genes for the changes of each gene in the pre-determined genome, according to the driving force information of each mutant gene in the several mutant genes for the changes of each gene in the pre-determined genome; and

S14: determining at least one pre-determined type of intracellular deterministic event of the detected object according to the driving force information of the several mutant genes for the changes of each gene in the pre-determined genome.

In one implementation, the determination of at least one pre-determined type of intracellular deterministic event of the detected object in S14 includes:

S141: acquiring a first type of intracellular deterministic event information of the detected object; and

S142: determining a second type of intracellular deterministic event information of the detected object according to the first type of intracellular deterministic event information of the detected object.

In this application, the detected object may be a living organism, for example, it may be but not limited to a human being.

Taking humans as an example, the pre-determined genome may be, for example, part or all of the genes in the known human genome.

The several mutant genes of the detected object belong to a pre-determined genome, which can be rare germline mutant genes or global germline mutant genes, depending on the actual situation.

In an implementation, global germline genetic information of the detected object can be obtained, such as whole exome sequencing data, from which rare germline mutant genes can be determined. In which, the rare germline mutant genes of the detected object can be determined, for example, by determining whether the mutant genes in the whole exome sequencing data of the detected object is in a pre-determined rare mutant genome. Rare germline mutant genomes can be determined by the set mutation frequency threshold. In other words, if the probability of a gene mutating in the population is greater than the set mutation frequency threshold, the gene is a rare germline mutant gene.

It can be understood that in other implementations, other Qualcomm global data can also be used to replace the whole exome sequencing data. The Qualcomm global data includes, but is not limited to, whole exome sequencing, whole genome sequencing, gene chips, and expression chip data, etc..

In one particular instance, the aforementioned first type of intracellular deterministic event information may be the driving force information of the several mutant genes of the detected object for changes in the activity of at least one pre-determined signaling pathway, and the second type of intracellular deterministic event information may be the predicted risk of developing a specific disease for the detected object.

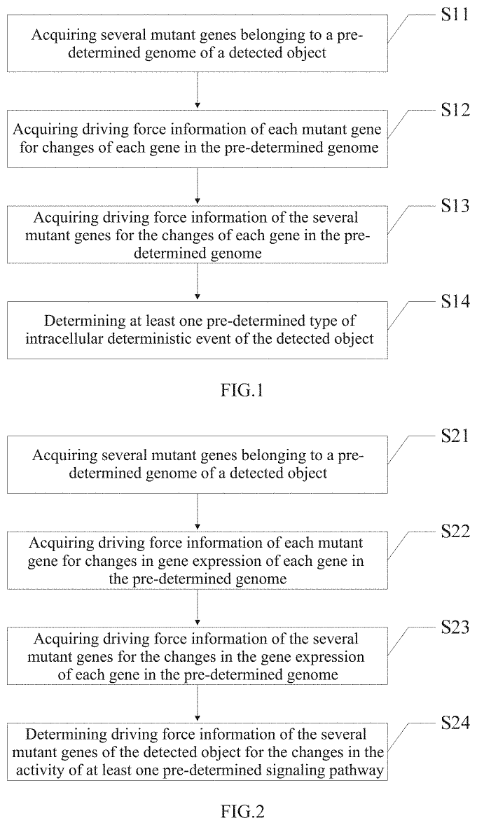

FIG. 2 shows a schematic flowchart of a method for acquiring an intracellular deterministic event according to an embodiment of the present application, and the method may be executed by an electronic device. In this embodiment, the driving force for the several mutant genes of the detected object to change the activity of at least one pre-determined signaling pathway can be obtained. The method of this embodiment includes:

S21: acquiring several mutant genes belonging to a pre-determined genome of a detected object;

S22: acquiring driving force information of each mutant gene in the several mutant genes for changes in gene expression of each gene in the pre-determined genome;

S23: acquiring driving force information of the several mutant genes for the changes in the gene expression of each gene in the pre-determined genome, according to the driving force information of each mutant gene in the several mutant genes for the changes in the gene expression of each gene in the pre-determined genome; and

S24: determining driving force information of the several mutant genes of the detected object for the changes in the activity of at least one pre-determined signaling pathway according to the driving force information of the several mutant genes for the changes in the gene expression of each gene in the pre-determined genome.

In the present application, gene expression refers to the amount of RNA product transcribed by a detected gene on the genome or the amount of protein that can be translated. The amount of gene expression may be a value in a continuous range and may be obtained from existing data.

In an implementation of the present application, the intracellular deterministic event information of at least one pre-determined type of the detected object includes: determining the driving force information of the several mutant genes of the detected object for the changes in the activity of a plurality of pre-determined signaling pathways. The plurality of pre-determined signaling pathways may be selected and determined from the existing signaling pathways in prior arts. When selecting, for example, a signaling pathway whose overlap of the genes contained in the signaling pathway and the genes in the pre-determined genome is greater than a pre-determined threshold may be selected.

The driving force for the mutant genes to change the activity of the signaling pathway indicates the ability of the mutant genes to influence the changes in the activity of the signaling pathway.

In an implementation of the present application, the step S22 of acquiring driving force information of each mutant gene in the several mutant genes for changes in the gene expression of each gene in the pre-determined genome includes:

Acquiring from pre-obtained template data, driving force information of each mutant gene in the several mutant genes for changes in the gene expression of each gene in the pre-determined genome, in which the template data includes the driving force information of each gene in the pre-determined genome for the changes in the gene expression of each gene in the pre-determined genome.

In an implementation of the present application, the method for acquiring the template data includes: performing the following processing for each gene gi in the pre-determined genome:

S221: dividing pre-determined reference cell lines into a first cell line group and a second cell line group, in which the first cell line group includes reference cell lines including the mutant gene gᵢ among the pre-determined reference cell lines, and the second cell line group includes reference cell lines that do not include the mutant gene gᵢ among the pre-determined reference cell lines.

S222: for each gene gⱼ in the pre-determined genome, acquiring difference information between a mean gene expression information of the mutant gene gⱼ of the reference cell line in the first cell line group and a mean gene expression information of the mutant gene g, of the reference cell line in the second cell line group.

S223: performing noise reduction processing on the difference information.

The following is a specific example for illustration.

Suppose the number of genes in the pre-determined genome is n, and the number of reference cell lines is p,

For each gene gᵢ in the pre-determined genome, p reference cell lines are divided into two groups: the first cell line group (also called a mutant group) mti and the second cell line group (also called a wild group) wti. In which, the first cell line group includes reference cell lines including the gene gᵢ among the p reference cell lines (set the number as pi1), and the second cell line group includes reference cell lines that do not include the gene gᵢ (set the number as pi2) among the p reference cell lines.

Then for each gene g, in the pre-determined genome, calculating the difference information between the mean gene expression information of the gene gⱼ of the pi1 reference cell line in the first cell line group and the mean gene expression information of the gene gⱼ of the pi2 reference cell line in the second cell line group; specifically, it may be calculating a mean difference de between a mean gene expression value of the gene gj of the pi1 reference cell line in the first cell line group and a mean gene expression value of the genes g, of the pi2 reference cell line in the second cell line group:

deᵢⱼ=μₘₜᵢⱼ−μ_{wtij}

In which, deᵢⱼ is the difference of the mean gene expression value of the gene gj of each reference cell line in the mutant group mti corresponding to the gene gi and the mean gene expression value of the gene gⱼ of each reference cell line in the wild group wti, μₘₜᵢⱼ denotes the mean gene expression value of the gene g, of each reference cell line in the mutant group mti, and μ_{wtij} denotes the mean gene expression value of the gene gⱼ of each reference cell line in the wild group wti.

Further, noise reduction processing may be performed on the above-mentioned difference deᵢⱼ.

In an implementation, a pre-determined number of random simulations (for example, but not limited to 10000 times) may be performed first. In each simulation, p cell lines were randomly divided into the mutant group and the wild group, and the number of reference cell lines in the mutant group was pi1, and the number of reference cell lines in the wild group was pi2. Then calculating the difference deₙᵤₗₗ of the mean expression values of each gene gi in the two groups randomly divided into two groups herein.

After that, performing a noise reduction processing on deᵢⱼ with the difference deₙᵤₗₗ obtained from each random simulation (also called standardization processing). The value acquired after the standardization processing represents the driving force df, which can be obtained by the following formula:

$${df}_{ij} = \frac{{de}_{ij} - mean ( {de}_{null} )}{std ( {de}_{null} )}$$

In which, dfᵢⱼ is the driving force information of gene gᵢ for the changes in the gene expression of gene gⱼ mean (deₙᵤₗₗ) and std (deₙᵤₗₗ) are the mean and standard deviation of deₙᵤₗₗ calculated by 10000 random simulations, respectively.

The above process is to calculate the driving force for a gene gᵢ to change the gene expression of each gene gⱼ. For the n genes in the pre-determined genome, the above calculation process is performed to obtain the driving force information of each gene in the pre-determined genome for the changes in the gene expression of each gene in the pre-determined genome, that is, the template data. In one implementation, the template data may be represented by an n x n matrix. Each row of the matrix corresponds to a gene gᵢ, and each column corresponds to a gene gⱼ. Each value in the matrix represents the driving force for the gene of the row to change the gene expression of the gene of the column.

Each detected object carries a different number of mutant genes. It is assumed that the detected object carries in mutant genes. In an implementation manner, determining the driving force information for each mutant gene in the in mutant genes of the detected object to change the gene expression of each gene in the pre-determined genome may include: acquiring in rows of data corresponding to the in mutant genes from the aforementioned n×n matrix, and a matrix of in x n can be obtained.

In an implementation of the present application, the step S23 of acquiring the driving force information for the several mutant genes of the detected object to change the gene expression of each gene in the pre-determined genome includes: performing the following processing for each gene gⱼ in the pre-determined genome:

S231: performing weighted mean processing on the driving force information of each of the several mutant genes of the detected object for the changes in the gene expression of each gene in the pre-determined genome.

In order to determine the overall effect of the in mutant genes of the detected object, the driving force of each gene can be weighted (w), and then the mean DF can be calculated.

$${DF}_{j} = \frac{{\sum }_{k = 1}^{m} w * {df}_{{i}_{k} j}}{m}$$

In which, DFⱼ is the mean of the driving force for all m mutant genes of the detected object to change the gene expression of the gene gⱼ in the pre-determined genome, ik denotes the number of rows in the n x n matrix of the k-th mutant genes of the detected object, df is the value of the corresponding position in the aforementioned n×n matrix.

A simple method is to assume that the weight of the driving force of each mutant gene is the same. It should be understood that the weight of the driving force of each mutant gene can also be different.

S232: Perform noise reduction processing on the result DFⱼ obtained by the weighted mean processing. In an implementation, a pre-determined number of random simulations (for example, but not limited to 10000 times) may be performed first. In each simulation, randomly select in genes from n genes in the pre-determined genome to perform weighted mean processing to obtain DFₙᵤₗₗ.

After that, the weighted mean DFₙᵤₗₗ obtained by each random simulation is used to perform noise reduction processing (also called standardization processing) on DFⱼ. This standardization processing can be obtained by the following formula:

$${ZDF}_{j} = \frac{{DF}_{j} - mean ( {DF}_{null} )}{std ( {DF}_{null} )}$$

ZDFⱼ represents the driving force for all in mutant genes carried by the detected object to change the gene expression of the gene gⱼ in the pre-determined genome, mean (DFₙᵤₗₗ) and std (DFₙᵤₗₗ) are the mean and standard deviation of DFₙᵤₗₗ calculated by 10000 random simulations, respectively.

After acquiring the driving force of all m mutant genes carried by the detected object to change the gene expression of each gene in the pre-determined genome, a matrix of 1×n is obtained. Although each detected object carries a different number of mutant genes, through the above processing, different m×n matrices corresponding to different detected objects are converted into the same 1×n matrix, which can be compared in the same dimension later.

In an implementation of the present application, assuming that a number of pre-determined signaling pathways is q, the acquiring the driving force information of the several mutant genes of the detected object for changes in the activity of at least one pre-determined signaling pathway in S24 includes: performing the following processing for each signaling pathway sⱼ:

S241: acquiring information about the influence of each gene gᵢ in the pre-determined genome on the activity of the signaling pathway sⱼ; and

S242: acquiring comprehensive influence information of the several mutant genes of the detected object on the activity of the signaling pathway sⱼ, according to the information about the influence of each gene gᵢ in the pre-determined genome on the activity of the signaling pathway sⱼ.

In an implementation of the present application, the acquiring information about the influence of each gene gᵢ in the pre-determined genome on the activity of the signaling pathway sⱼ in S241 includes:

S2411: acquiring driving force information of each gene gᵢ for changes in the gene expression of each gene in the signaling pathway sⱼ;

S2412. acquiring influence information of the change in gene expression of each gene ak in the signaling pathway sⱼ on the signaling pathway sⱼ; and

S2413: acquiring influence information of each gene gᵢ in the pre-determined genome on the activity of the signaling pathway sⱼ according to the driving force information acquired in S2411 and the influence information acquired in S2412.

In an implementation of the present application, firstly, information about the influence of each gene gᵢ in the pre-determined genome on the activity of the signaling pathway sⱼ is obtained. Assuming that a signaling pathway is composed of k genes, the change in gene expression of each gene ak in the signaling pathway has two effects on the activity of the signaling pathway, namely, up-regulation (up) or down-regulation (down), then the influence of gene gᵢ on the activity of the j-th signaling pathway can be determined by the following formula:

$${DFP}_{ij} = {\sum }_{a = 1}^{k} {df}_{{ij}_{a}} * {sig}_{a}$$

$${sig}_{a} = \{ \begin{matrix}- 1 , & down \\ 1 , & up\end{matrix}$$

In which, DFPᵢⱼ is an influence value of a gene gᵢ in the pre-determined genome on the activity of the j-th signaling pathway, df is a value of the corresponding position in the aforementioned n×n matrix, and jₐ is the number of column of the a-th gene in the j-th signaling pathway in the n×n matrix; sigₐ denotes the influence of the a-th gene ak on the activity of the j-th signaling pathway, which can be acquired from the existing data. In one example, the value of up-regulation is 1 and the value of down-regulation is −1.

Moreover, DFPᵢⱼ can be subjected to noise reduction processing.

In an implementation, a pre-determined number of random simulations (for example, but not limited to 10000 times) may be performed first. In each simulation, data corresponding to k genes can be randomly selected from the aforementioned n×n matrix to calculate DFPₙᵤₗₗ by the above formula.

After that, use the DFPₙᵤₗₗ obtained in each random simulation to perform noise reduction processing (also known as standardization) on DFP. This standardization processing can be determined by the following formula:

$${ZDFP}_{ij} = \frac{{DFP}_{ij} - mean ( {DFP}_{null} )}{std ( {DFP}_{null} )}$$

In which, ZDFPᵢⱼ is the driving force for a gene gi in the pre-determined genome to change the activity of the j-th signaling pathway, mean (DFPₙᵤₗₗ) and std (DFPₙᵤₗₗ) are the mean and standard deviation of DFPₙᵤₗₗ calculated by 10000 random simulations, respectively.

After acquiring the driving force ZDFPᵢⱼ for each gene gᵢ of the n genes in the pre-determined genome to change the activity of each of the q pre-determined signaling pathways, a matrix of n×q can be obtained.

In an implementation of the present application, the comprehensive influence information of the several mutant genes of the detected object on the activity of the signaling pathway sⱼ in S242 can be obtained by the following formula:

$${IDFP}_{j} = \frac{{\sum }_{a = 1}^{m} {ZDFP}_{{i}_{a} j}}{m}$$

In which, IDFPⱼ is the comprehensive influence of the in mutant genes of the detected object on the activity of the signaling pathway sⱼ, and iₐ is the number of rows of the a-th gene in the j-th signaling pathway in the aforementioned n×60 matrix.

Further, IDFPⱼ can be subjected to noise reduction processing.

In an implementation manner, a pre-determined number of random simulations (for example, but not limited to 10000 times) may be performed first. In each simulation, randomly select in rows from the n×60 matrix to calculate IDFPₙᵤₗₗ through the above formula.

After that, the IDFPₙᵤₗₗ obtained in each random simulation is used to perform noise reduction processing (also known as standardization) on IDFPⱼ. This standardization can be determined by the following formula:

$${ZIDFP}_{j} = \frac{{IDFP}_{j} - mean ( {IDFP}_{null} )}{std ( {IDFP}_{null} )}$$

In which, ZIDFPⱼ is the driving force for all in mutant genes carried by the detected object to change the activity of the j-th signaling pathway, mean(IDFPₙᵤₗₗ) and std(IDFPₙᵤₗₗ) are the mean and standard deviation of IDFPₙᵤₗₗ calculated by 10000 random simulations, respectively.

After acquiring the driving force for all in mutant genes carried by the detected object to change the activity of each signaling pathway, a matrix of 1×q can be obtained. In this way, each detected object is represented by a 1×q matrix, without considering the mutant gene data and specific mutant genes of the detected object.

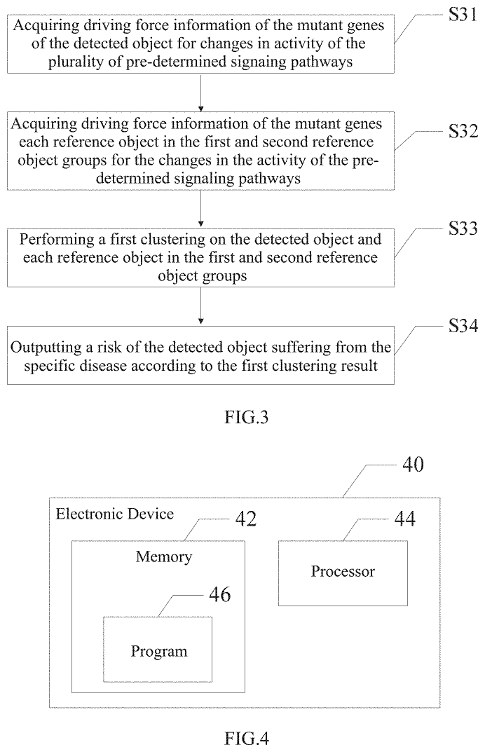

FIG. 3 shows a schematic flowchart of a method for predicting a risk of suffering from disease according to an embodiment of the present application. The method may be executed by an electronic device and includes:

S31: acquiring driving force information of the mutant genes belonging to the pre-determined genome of the detected object for changes in the activity of the plurality of pre-determined signaling pathways;

S32: acquiring driving force information of the mutant genes belonging to the pre-determined genome of each reference object in the first and second reference object groups for the changes in the activity of the pre-determined signaling pathways; in which, each reference object in the first reference object group belongs to a healthy object, and each reference object in the second reference object group belongs to an object suffering from a specific disease;

S33: performing a first clustering on the detected object and each reference object in the first and second reference object groups, according to the driving force information of the mutant genes of the detected object for the changes in the activity of the plurality of pre-determined signaling pathways, and the driving force information of the mutant genes of each reference object in the first and second reference object groups for the changes in the activity of the plurality of pre-determined signaling pathways; and

S34: outputting a risk of the detected object suffering from the specific disease according to the first clustering result acquired after performing the first clustering.

In a specific example, the specific disease may be triple negative breast cancer. It should be understood that the method for predicting a risk of suffering from disease of this embodiment can also be used for other suitable specific diseases, and is not limited to triple-negative breast cancer.

In an implementation, after performing the first clustering on the detected object and each reference object in the first and second reference object groups, the method further includes combining the plurality of clusters obtained after performing the first clustering into multiple groups.

In an implementation, after performing the first clustering on the detected object and each reference object in the first and second reference object groups, the method further includes acquiring and outputting at least one of clinical or pathological related deterministic event characteristics, pathological characteristics, physiological characteristics, and behavioral characteristics of the reference object belonging to the same disease risk level as the detected object.

In an implementation, the NMRCLUST clustering method is used to perform the first clustering on the detected object and each reference object in the first and second reference object groups. It can be understood that other clustering methods can be selected for the first clustering according to actual conditions. For example, including but not limited to hierarchical methods (such as k-nearest-neighbor (referred to as kNN) algorithms, etc.), Partition-based methods (such as K-Means clustering, etc.), Density-based methods (such as Density-Based Spatial Clustering of Applications with Noise ((Referred to as DBSCAN, etc.)), Grid-based methods (such as Statistical Information Grid (referred to as STING) algorithm, etc.), or Model-based methods (such as Gaussian Mixture Models, (referred to as GMM,)) etc., the present application includes but is not limited to this.

In an implementation, before acquiring the driving force information of the mutant genes of the detected object for the changes in the activity of the plurality of pre-determined signaling pathways, the method further includes: determining the plurality of pre-determined signaling pathways from multiple reference signaling pathways

In an implementation manner, determining the pre-classification type corresponding to the detected object includes: acquiring driving force information of the mutation gene of the detected object for the changes in activity of the multiple reference signaling pathways; acquiring driving force information of the mutant gene of each reference object in the third and fourth reference object groups for the changes in activity of the multiple reference signaling pathways; and performing a second clusteringon each reference object in the detected object, the third and fourth reference object groups according to the driving force information of the mutation gene of the detected object for the changes in activity of the multiple reference signaling pathways and the driving force information ofthe mutant gene of each reference object in the third and fourth reference object groups for the changes in activity of the multiple reference signaling pathways.

In an implementation manner, the Ward Hierarchical Clustering method is used to perform the second clustering on each reference object in the detected object and the third and fourth reference object groups.It can be understood that other clustering methods can be selected for the second clustering according to actual conditions. For example,Hierarchical methods (such as k-nearest-neighbor (referred to as kNN) algorithm, etc.), Partition-based methods (such as K-Means clustering, etc.),Density-based methods (such as Density-Based Spatial Clustering of Applications with Noise (abbreviated as DBSCAN) Etc.),Grid-based methods (such as STatistical INformation Grid (referred to as STING) algorithm, etc.), or Model-based methods (such as Gaussian Mixture Models, referred to as For GMM)) etc. can also be used, the present application includes but is not limited to this.

In an implementation manner of the present application,determining the several predetermined signaling pathways from a plurality of reference signaling pathways according to the pre-classification type includes: determining a fifth reference object group corresponding to the pre-classification typefrom the third reference object group according to the pre-classification type;determininga sixth reference object group corresponding to the pre-classification typefrom the fourth reference object group according to the pre-classification type; for each signaling pathway sk in the plurality of signaling pathways, determining a difference between the driving force information of the mutant gene of each reference object in the fifth reference object group for the changes in activity of the signaling pathway sk and the driving force information of the mutant gene of each reference object in the sixth reference object group for the changes in activity of the signaling pathway sk; and determining the plurality of predetermined signaling pathways that meet the preset difference significance condition from the plurality of information paths according to the difference.

In an implementation manner of the present application, the method for determininga difference between the driving force information of the mutant gene of each reference object in the fifth reference object group for the changes in activity of the signaling pathway sk and the driving force information of the mutant gene of each reference object in the sixth reference object group for the changes in activity of the signaling pathway sk includes: determining a difference between the average driving force value of the mutant gene of each reference object in the fifth reference object group for the changes in activity of the signaling pathway sk and the average driving force value of the mutant gene of each reference object in the sixth reference object group for the changes in activity of the signaling pathway sk.

Further, noise reduction processing can be performed on the difference.

In an implementation manner of the present application, outputting the risk of the detected object suffering from the specific disease according to the first clustering result obtained after performing the first clustering includes: determining and outputting the risk of the subject to the specific disease at least according to the cluster to which the detected object belongs and the ratio of the number of reference objects belonging to the second reference object group in the cluster and the number of reference objects belonging to the first reference object group.

In the following, taking triple-negative breast cancer as an example, a specific example is used to illustrate the disease risk prediction method of the present application in detail. In the embodiment, the driving force information of the plurality of mutant genes of the detected object obtained in the embodiment of the method for acquiring intracellular deterministic events to change the activity of q predetermined signaling pathways can be used to predict the risk of triple-negative breast cancer for the subject.

In the application,triple negative breast cancer (TNBC) refers to estrogen receptor (ER), progesterone receptor (PR), HER2 genesdetected in the molecular typing of breast cancer are all negativeBreast cancers, and account for about 15% of all breast cancer patients, and have the characteristics of early onset, poor prognosis, unclear pathogenesis, and low response to treatment.

For the third reference object group consisting of n₁ healthy people, each person can be represented by the aforementioned 1×q matrix, which represents the driving force information of the mutant gene of each person for the changes in activity of q signaling pathways. Clustering analysis of these ma 1×q matrices, that is, n₁×q matrices (for example, analysis by the Ward Hierarchical Clustering method), found that these reference objects can be divided into two types: A and B.

For the fourth reference group consisting of n₂ triple-negative breast cancer patients, each patient can be represented by the aforementioned 1 xq matrix, which represents the driving force information of the mutant gene of each person for the changes in activity of q signaling pathways. Clustering analysis of these n₂ of 1×q matrices, that is, n2×q matrices (for example, analysis by the Ward Hierarchical Clustering method), found that these people can also be divided into two types: A and B.

In other words, performing clustering analysis on the n₁ xq matrices and the n₂ xq matrices corresponding to the third reference object group and the fourth reference object group, and the reference objects in the third and fourth reference object groups can be divided intotypes A and B,andboth types include healthy people and triple-negative breast cancer patients.

When it is necessary to predict the risk of the detected object suffering from the triple-negative breast cancer, 1×q matrix of the detected object can be obtained according to the method in the foregoing embodiment.Then, the 1 xq matrix of the detected object is combined with the n₁ xq matrix and the n₂ xq matrix corresponding to the third and fourth reference object groups to perform a second clustering, for example, by Ward Hierarchical Clustering, to determine the pre-classification type of the detected object. As mentioned above, the reference objects in the third and fourth reference object groups will be divided into types A and B, the detected objects will be clustered into type A or type B, that is, after the second clustering, it can be determined that the pre-classification type of the detected object is type A or type B.

Assuming that the pre-classification type of the detected object is the type A, the fifth reference object group corresponding to the type A is determined from the third reference object group, and the sixth reference object group corresponding to the type A is determined from the fourth reference object group. R It is understandable that the fifth reference object group may include part or all of the reference objects of type A in the third reference object group, and the sixth reference object group may include some or all of the type A reference objects in the fourth reference object group.Assuming that the number of healthy persons of type A in the fifth reference object group and the number of triple-negative breast cancer patients of type A in the sixth reference object group are mₐ and n₂ₐ, respectively, then the difference DPₖ between the driving force information of the mutant gene of each triple-negative breast cancer patient of type A in the sixth reference group for the changes in activity of the k-th signaling pathway sk and the driving force information of the mutant gene of eachhealthy person of type A in the fifth reference group for the changes in activity of the k-th signaling pathway sk can be determined by the following formula:

$${DP}_{k} = \frac{{\sum }_{i = 1}^{{n}_{2 a}} {ZIDFP}_{ik}}{{n}_{2 a}} - \frac{{\sum }_{j = 1}^{{n}_{1 a}} {ZIDFP}_{jk}}{{n}_{1 a}}$$

Among them, ZIDFPᵢₖ is the driving force of the mutated gene carried by the i-th triple-negative breast cancer patient for the changes in activity of the k-th signaling pathway; ZIDFPjk is the driving force of the mutated gene carried by the j-th healthy person for the changes in activity of the k-th signaling pathway.

Among them, ZIDFPik is the driving force of the mutated gene carried by the i-th triple-negative breast cancer patient on the activity of the k-th signaling pathway, and ZIDFPjk is the effect of the mutant gene carried by the j-th healthy person on the activity of the k-th signaling pathway.

Further, DPₖ can be processed for noise reduction.

In an implementation manner, a predetermined number of random simulations (for example, but not limited to 1,000,000 times) may be performed first. In each random simulation, the label of each reference object is a healthy person or a triple-negative breast cancer patient is randomly shuffled, and DPₙᵤₗₗ can be calculated according to the above formula.

After that, use the DPₙᵤₗₗ obtained in each random simulation to perform noise reduction processing (also known as standardization) on DPₖ. This standardization can be achieved by the following formula:

$${ZDP}_{k} = \frac{{DP}_{k} - mean ( {DP}_{null} )}{std ( {DP}_{null} )}$$

Among them, mean (DPₙᵤₗₗ) and std (IDFPnull) are the average and standard deviation of DPₙᵤₗₗ calculated by 1,000,000 random simulations, respectively. The more ZDPₖ deviates from 0, it means that the difference in the activity of this signaling pathway between triple-negative breast cancer patients and healthy people is not random, but has specific biological significance.

Then, it can determine the several signaling pathways that meet the pre-set difference significance condition from the q information pathways according to the obtained difference between the driving force information of the mutant gene of each reference object in the fifth reference object group for the changes in activity of the q signaling pathways and the driving force information of the mutant gene of each reference object in the sixth reference object group for the changes in activity of the q signaling pathways.

In an implementation manner, q1 (for example, 8) signaling pathways with the largest absolute value of ZDPₖ among the q signaling pathways may be selected for subsequent analysis.

The q1 row data corresponding to the ql signaling pathway is obtained from the 1×q matrix of the detected object, and the driving force information of the mutation gene of the detected object for the changes in activity of the ql reference signaling pathway is obtained.

In addition, the pre-classification type of the detected object is type A, the first reference object group corresponding to healthy people of type A is determined from the third reference object group, and the second reference object group corresponding totriple-negative breast cancerof type Ais determined from the fourth reference object group. The ql row data corresponding to the ql signaling pathway are respectively obtained from the lx q matrix of each reference object in the first and second reference object groups, and the driving force information of the mutant gene of each reference object in the first and second reference object groups for the changes in activity of the q1 reference signaling pathway.

It is understandable that the first reference object group may include part or all of the reference objects of type A in the third reference object group, and the second reference object group may include part or all of the reference objects of type A in the fourth reference object group. The first reference object group may be the same as or different from the fifth reference object group, and the second reference object group may be the same as or different from the sixth reference object group.

Subsequently, performing the first clustering on the detected object and each reference object in the first and second reference object groups to obtain ul clusters according to the driving force information of the mutant gene of the tested object for the changes in activity of the ql reference signaling pathway and the driving force information of the mutant gene of each reference object in the first and second reference object groups for the changes in activity of the ql reference signaling pathway.

The first clustering can be implemented using the NMRCLUST clustering method, for example. The NIVIRCLUST clustering method uses average link distance clustering, and then uses a penalty function to optimize the number of clusters and the distance between clusters at the same time. For example, the number of clusters corresponding to the minimum penalty value can be selected to cluster the detected object of typeA and each reference object in the first and second reference object groups into u (for example, 15) clusters, and each cluster can correspond to different risk levels of disease. It can be understood that other clustering methods can be selected to perform the first clustering according to actual conditions, and the present application is not limited to this.

Then, outputting the risk of the detected subject suffering from triple negative breast canceraccording to the first clustering result obtained after performing the first clustering. After the first clustering is performed, it can be determined which of the u clusters the detected object belongs to, and the number of reference objects belonging to the first reference object group (that is, the number of healthy people) and the number of reference objects belonging to the second reference object group (ie, the number of triple-negative breast cancer patients) in each cluster. Then calculating the percentage of the number of triple-negative breast cancer patients and the number of healthy people in each cluster, as a quantitative parameter characterization of the risk level of the disease, the larger the percentage value, the more likely to have triple-negative breast cancer. Sorting the percentages corresponding to each cluster by size can determine the level of disease risk corresponding to each cluster. Therefore, based on the cluster to which the detected object belongs, the risk of the detected object of triple-negative breast cancer can be predicted.

It is understandable that it is also possible to determine and output the risk of detected object suffering from triple-negative breast cancerdirectlyaccording to the cluster to which the detected object belongs and the ratio of the number of reference objects belonging to the second reference object group and the number of reference objects belonging to the first reference object group.

Further, when the number of clusters obtained by performing the first clustering is larger, the clusters obtained after performing the first clustering may be merged according to the data distribution characteristics, so as to obtain a group with more prominent characteristics. For example, the u disease risk levels are merged into a smaller number of disease risk levels, so as to facilitate the reference of the detected object.

In another implementation manner, the pre-classification type corresponding to the detected object may be determined by comparing the preset classification rules of various types with the information corresponding to the classification rule of the detected object.For example, in one example, each reference object in the aforementioned third reference object group and the fourth reference object group may be subjected to a second clustering, and the reference objects in the third and fourth reference object groups can be divided into types A and B, and then the relevant information of the reference object of type A and the reference object of type B (for example, the driving force information of the mutant gene of each person in the various reference objects for the changes in activity of the q signaling pathways) are calculated to obtain each classification rule of each type;when determining the pre-classification type corresponding to the detected object, the information corresponding to the classification rule of the detected object (for example, the driving force information of mutant gene of the detected object for the changes in activity of q signaling pathways)is compared with the classification rules of each type, and the detected objects are classified into the closest type in each type.It is understandable that the foregoing only gives a specific example of determining the pre-classification type corresponding to the detected object according to the preset classification rules of each type in the present application,and the present application is not limited to this.For example, in other embodiments, the classification rules of each type can be determined in other ways, and the information corresponding to the classification rules of the detected object is not limited to the exemplary information mentioned above.

In an implementation of the present application, in addition to outputting the predicted risk of the detected object suffering from triple-negative breast cancer,it can also obtain and output the clinical or pathologically relevant deterministic event characteristics (such as age of onset, lymph node metastasis, etc.), pathological characteristics (such as drug response, primary or metastatic, etc.), physiological characteristics (immune function, cardiovascular and respiratory system functions, etc.), and behavioral characteristics (such as diet and exercise, etc.) of reference objects belonging to the same disease risk level as the detected object (for example, the same cluster or the same group).

It is understandable that the presentapplication is described above by taking triple-negative breast cancer as an example, but the present application does not limit that pre-classification must be performed, or the pre-classification types are limited to only two types. In other embodiments of the present application, for example, in the method for predicting the risk of other diseases, the pre-classification types may be more than two, or pre-classification may not be required.

FIG. 4 shows an electronic device 40 according to an embodiment of the present application, including a memory 42, a processor 44, and a program 46 stored in the memory 44, the program 46 is configured to be executed by the processor 44, and the processor 44 executes the program implements at least part of the aforementioned method for acquiring intracellular deterministic event, or implements at least part of the aforementioned method for predicting risk of disease, or a combination of the two methods.

In some embodiments of the present application, the germline genetic information that can be collected during the asymptomatic period is used to obtain intracellular deterministic event through the driving force information of the mutant gene of the detected object for changing the gene in the genome.

In some embodiments of the present application, all germline genetic information are used to comprehensively evaluate the basis of the overall characteristics of germline inheritance, so that it can cover the risks evaluation of various sporadic and familial genetic diseases (such as breast cancer) caused by germline inheritance, and the sensitivity of detecting individuals at risk is improved.

In some embodiments of the present application, germline variation features with discrete, high-dimensional, multi-correlated, and non-standardized can be projected to gene prediction expression features and signaling pathway activity features with continuous range, relatively low-dimensional, and gradually converging correlation, it constructs a quantitative model that converts discrete qualitative data into continuous space, on the one hand, it retains the globalfeatures of the data, on the other hand, it becomes the basis of data-driven classification that correlates germline genetic information with other deterministic events in breast cancer (including but not limited to lymph node metastasis, age of onset and other pathophysiological characteristics).

In some embodiments of the present application, since the input source is a global germline rare mutation, the risk rating and clinical feature correlation of sporadic genetic breast cancer such as triple-negative breast cancer can be graded according to pathway activity, which fills up the gap in the coverage of the knowledge-driven approach based on gene panel and significantly reduces the false negative rate.

In some embodiments of the present application, the risk of disease can be correlated with other clinical, pathological, physiological, or behavioral related deterministic event features, so that the model can provide a basis for prognostic evaluation, early clinical intervention and management of patients based on germline genetic information.

The electronic device may be a user terminal device, a server, or a network device in some embodiments. For example, mobile phones, smart phones, laptops, digital broadcast receivers, personal digital assistants (PDAs), PAD (tablet computers), portable multimedia player (PMP), navigation devices, in-vehicle devices, digital TVs, desktop computers, etc., single A network server, a server group composed of multiple network servers, or a cloud composed of a large number of hosts or network servers based on cloud computing, etc.

The memory includes at least one type of readable storage medium, the readable storage medium includes flash memory, hard disk, multimedia card, card-type memory (such as SD or DX memory, etc.), random access memory (RAM), static random access memory (SRAM), read only memory (ROM), electrically erasable programmable read only memory (EEPROM), programmable read only memory (PROM), magnetic memory, magnetic disks, optical disks, etc. The memory stores the operating system and various application software and data installed in the service node device.

The processor may be a central processing unit (CPU), controller, microcontroller, microprocessor, or other data processing chip in some embodiments.

In the above-mentioned embodiments, the description of each embodiment has its own emphasis. For parts that are not described in detail or recorded in an embodiment, reference may be made to related descriptions of other embodiments.

Those skilled in the art may be aware that the units and algorithm steps of the examples described in combination with the embodiments disclosed herein can be implemented by electronic hardware or a combination of computer software and electronic hardware. Whether these functions are executed by hardware or software depends on the specific application and design constraint conditions of the technical solution. Professionals and technicians can use different methods for each specific application to implement the described functions, but such implementation should not be considered as going beyond the scope of the present application.

A whole or part of flow process of implementing the method in the aforesaid embodiments of the present application can also be accomplished by using computer programto instruct relevant hardware. When the computer program is executed by the processor, the steps in the various method embodiments described above can be implemented. Wherein, the computer program comprises computer program codes, which can be in the form of source code, object code, executable documents or some intermediate form, etc. The computer readable medium can include: any entity or device that can carry the computer program codes, recording medium, USB flash disk, mobile hard disk, hard disk, optical disk, computer storage device, ROM (Read-Only Memory), RAM (Random Access Memory), electrical carrier signal, telecommunication signal and software distribution medium, etc. It needs to be explained that, the contents contained in the computer readable medium can be added or reduced appropriately according to the requirement of legislation and patent practice in a judicial district, for example, in some judicial districts, according to legislation and patent practice, the computer readable medium doesn't include electrical carrier signal and telecommunication signal.

As stated above, the aforesaid embodiments are only intended to explain but not to limit the technical solutions of the present application. Although the present application has been explained in detail with reference to the above-described embodiments, it should be understood for the ordinary skilled one in the art that, the technical solutions described in each of the above-described embodiments can still be amended, or some technical features in the technical solutions can be replaced equivalently; these amendments or equivalent replacements, which won't make the essence of corresponding technical solution to be broken away from the spirit and the scope of the technical solution in various embodiments of the present application, should all be included in the protection scope of the present application.

1. A method for acquiring intracellular deterministic event, executed by an electronic device, comprising:
S11: acquiring several mutant genes of a detected object;
S12: acquiring driving force information of each of the several mutant genes for changes of each gene in a predetermined genome;
S13: acquiring driving force information of the several mutant genes for changes of each gene in the predetermined genome according to the acquiring driving force information of each of the several mutant genes for the changes of each gene in the predetermined genome; and
S14: determining at least onepredetermined type of intracellular deterministic event information of the detected object according to the driving force information of the several mutant genes for the changes of each gene in the predetermined genome.

2. The method of claim 1, wherein the determining at least onepredetermined type of intracellular deterministic event information of the detected object comprises:
acquiring a first type of intracellular deterministic event information of the detected object; and
determining a second type of intracellular deterministic event information of the detected object according to the first type of intracellular deterministic event information of the detected object.

3. The method of claim 1, wherein the determining at least onepredetermined type of intracellular deterministic event information of the detected object comprises:
determining driving force information of the several mutant genes for changing an activity of at least one predetermined signal pathway.

4. The method of claim 1, wherein the acquiring driving force information of each of the several mutant genes for changes of each gene in the predetermined genome comprises:
acquiring driving force information of each ofthe several mutant genesfrom pre-obtained template data for changing gene expression of each gene in the predetermined genome, wherein the template data comprises driving force information of each mutant gene in the predetermined genome for changing gene expression of each gene in the predetermined genome.

5. The method of claim 4, wherein the method for acquiring the template data comprises: performing each gene gi in the predetermined genome as following:
dividing a predetermined reference cell line into a first cell line group and a second cell line group, wherein the first cell line group comprises a reference cell line in the predetermined reference cell line that includes the gene gi, and the second cell line group comprises a reference cell line in the predetermined reference cell line that does not include the gene gi; and
for each gene gj in the predetermined genome, acquiring difference information between average gene expression information of the gene gj of the reference cell line in the first cell line group and average gene expression information of the gene gj of the reference cell line in the second cell line group.

6. The method of claim 5, wherein the method of acquiring the template data further comprises:
performing a noise reduction processing on the difference information.

7. The method of claim 1, wherein in the S13, the method for acquiring driving force information of the several mutant genes for changing the gene expression of each gene in the predetermined genome comprises:
for each mutant gene gj in the predetermined genome, performing weighted average processing onto the driving force information of each mutant gene in the several mutant genes for changing the gene expression of each gene in the predetermined genome.

8. The method of claim 1, wherein in the S13, the method for acquiring driving force information of the several mutant genes for changing the gene expression of each gene in the predetermined genome further comprises:
performing a noise reduction processing on a result obtained by the weighted average processing.

9. The method of claim 3, wherein in the S14, the acquiring the driving force information of the several mutant genes for changing the activity of at least one predetermined signal pathway comprises: performing each of the signal pathways as following:
acquiring influence information of each gene gi in the predetermined genomeon the activity of the signal pathway sj; and
acquiring comprehensive influence information of the several mutant genes of the detected object on the activity sj of the signal pathway sj according to the influence information of each gene gi in the predetermined genome on the activity of the signal pathway sj.

10. The method of claim 9, wherein the acquiring influence information of each gene gi in the predetermined genome on the activity of the signal pathway sj comprises:
S2411: acquiring driving force information of each gene gi for changing gene expression of each gene a in the signal pathway sj;
S2412: acquiring influence information of the change of gene expression of each gene a in the signal pathway sj on the signal pathway sj; and
S2413: acquiring influence information of each gene gi in the predetermined genome on the activity of the signal pathway sj according to the driving force information obtained in S2411 and the influence information obtained in S2412.

11. The method of claim 3, wherein the determining at least one predetermined type of intracellular deterministic event information of the detected object comprises: determining driving force information of the several mutant gene of the detected object to the changes in activity of a plurality of predetermined signal pathways; wherein a degree of overlap between genes contained in each signal pathway of the plurality of predetermined signal pathways and genes in the predetermined genome is greater than a predetermined threshold.

12. The method of claim 2, wherein the first type of intracellular deterministic event information is driving force information of the several mutant genes of the detected object to the changes in activity of a plurality of predetermined signal pathway, and the second type of intracellular deterministic event information is a predicted risk of the detected object suffering from a specific disease.

13. An electronic device, comprising: a memory, a processor, and a program stored in the memory, the program is configured to be executed by the processor, and when the processor executes the program to implement steps as following:
S11: acquiring several mutant genes of a detected object;
S12: acquiring driving force information of each of the several mutant genes for changes of each gene in a predetermined genome.
S13: acquiring driving force information of the several mutant genes for changes of each gene in the predetermined genome according to the acquiring driving force information of each of the several mutant genes for the changes of each gene in the predetermined genome; and
S14: determining at least one predetermined type of intracellular deterministic event information of the detected object according to the driving force information of the several mutant genes for the changes of each gene in the predetermined genome.

14. A storage medium, storing a computer program, wherein when the computer program is executed by a processor, to implement steps as following:
S11: acquiring several mutant genes of a detected object;
S12: acquiring driving force information of each of the several mutant genes for changes of each gene in a predetermined genome;
S13: acquiring driving force information of the several mutant genes for changes of each gene in the predetermined genome according to the acquiring driving force information of each of the several mutant genes for the changes of each gene in the predetermined genome; and
S14: determining at least one predetermined type of intracellular deterministic event information of the detected object according to the driving force information of the several mutant genes for the changes of each gene in the predetermined genome.
